(19) United States
(12) Patent Application Publication
(10) Pub. No.: US 2026/0232921 A1
(43) Pub. Date: Aug. 13, 2026

(54) MEDICAMENT DELIVERY DEVICE

(71) Applicant: SHL Medical AG, Zug (CH)

(72) Inventor: Joakim Gunnar Lindholm, Nacka Strand (SE)

(21) Appl. No.: 19/148,131

(22) PCT Filed: Dec. 13, 2023
(86) PCT No.: PCT/EP2023/085589

§ 371 Date: Jul. 15, 2025

(30) Foreign Application Priority Data
Jan. 24, 2023 (EP) 23152979.3

(51) Int. Cl. A61M 5/32; A61M 5/20

(52) U.S. Cl. CPC A61M 5/3245; A61M 5/20; A61M 5/3202; A61M 2005/2013; A61M 2005/2073

(57) ABSTRACT

The present disclosure relates to a medicament delivery device, the medicament delivery device having a proximal end pointing towards a dose delivery site during use of the medicament delivery device, and a distal end opposite the proximal end. The medicament delivery device includes a housing comprising a longitudinal axis. The medicament delivery device further includes a medicament delivery member cover is axially moveable within the housing between an extended position, a retracted position and a final position. The medicament delivery member cover includes at least two medicament delivery member cover locks disposed in a circumferential direction of the medicament delivery member cover and flexibly extendable in a radial outward direction with respect to the longitudinal axis. In the extended position and the retracted position, the flexible medicament delivery member cover locks are configured to abut an inner circumferential surface of the housing in a pre-tensioned state. In the final position after a medicament delivery, the medicament delivery member cover locks are configured to flex and extend in the radial outward direction. The housing comprises at least two relief pockets, wherein each relief pocket is provided in the outer circumferential surface of the housing at a certain distance from a proximal end of the housing, and each relief pocket is configured to receive at least a portion of the respective medicament delivery member cover lock.

### TECHNICAL FIELD

The present disclosure relates to a medicament delivery device and in particular to a lockout mechanism for a medicament delivery device after dose delivery.

### BACKGROUND

A medicament delivery device, in particular an autoinjector, according to the prior art comprises a housing comprising a front housing and a rear housing. The housing is adapted to hold a medicament container, such as a syringe. The syringe may be a pre-filled syringe and have a needle as a medicament delivery member arranged at a proximal end. When the autoinjector and/or the syringe are assembled, a protective needle sheath may be removably coupled to the needle. The protective needle sheath may be made of a flexible shell (for instance composed of rubber) and/or a rigid shell (for instance composed of plastic). A stopper, also called plunger, may be arranged for sealing the syringe distally and for expelling a medicament contained in the syringe through the needle. In other exemplary embodiments, the medicament container may be a cartridge which includes the medicament and engages a removable needle (e.g., by threads, snaps, friction, etc.).

A cap may be removably disposed at a proximal end of the housing. The cap may include an element (e.g., a barb, a hook, a narrowed section, etc.) arranged to engage the protective needle sheath, the housing and/or a medicament delivery member cover, also referred to as needle cover, telescoped within the housing. The cap may comprise grip features for facilitating removal of the cap (e.g., by twisting and/or pulling the cap relative to the housing).

A medicament delivery member cover biasing member (for instance a coil spring) may be arranged to bias the medicament delivery member cover in a proximal direction relative to the housing, before injection, for safety reasons. The medicament delivery member cover biasing member needs to be compressed at the beginning of injection in order to retract the medicament delivery member cover inside the housing and uncover the medicament delivery member, thereby activating the device.

A pre-loaded drive biasing member, for instance a pre-compressed drive coil spring, may be arranged within the housing. A plunger rod may be used to forward force from the drive biasing member to the stopper. In an exemplary embodiment, the plunger rod is hollow and the drive biasing member is arranged within the plunger rod. In another exemplary embodiment, the plunger rod is solid and the drive biasing member engages a distal end of the plunger rod. When the pre-loaded drive biasing member is released, the drive biasing member biases the plunger rod in the proximal direction relative to the housing, releasing the plunger rod. Besides that, the plunger rod may itself comprise means for sealing the medicament container such that no stopper needs to be provided.

A plunger rod release mechanism may be arranged for preventing release of the plunger rod prior to retraction of the medicament delivery member cover relative to the housing and for releasing the plunger rod only once the medicament delivery member cover is sufficiently retracted.

A medicament delivery member cover lock mechanism (needle cover lock mechanism) may be arranged to prevent retraction of the medicament delivery member cover relative to the housing when the cap is in place, thereby avoiding unintentional activation of the autoinjector (e.g., if dropped, during shipping or packaging, etc.). Such mechanisms are known in the art and will not be described in detail.

In addition to the medicament delivery member lock mechanisms preventing unintentional activation, in some devices, locking means are provided blocking the needle cover in an extended position after medicament delivery such that a user cannot easily or accidentally reach the needle. However, the locking means of the prior art may be subject to fatigue, creep or designed such that the protection may be easily bypassed, intentionally or unintentionally.

In particular, the locking mechanisms are generally manufactured of plastics and are thus subject to fatigue, creep or other changes in material properties during their lifespan and particular during storage periods.

Thus, there is a need to improve safety of medicament delivery devices and in particular the locking mechanisms of the needle cover after medicament delivery and ensure correct functioning also after long storage periods.

This need is met by a device according to the present disclosure.

### SUMMARY

Reference should now be made to the appended claims.

The present disclosure relates to a medicament delivery device, the medicament delivery device having a proximal end pointing towards a dose delivery site during use of the medicament delivery device, and a distal end opposite the proximal end. The medicament delivery device comprises a housing comprising a longitudinal axis, a medicament delivery member cover axially moveable within the housing between an extended position, a retracted position and a final position, the medicament delivery member cover comprising at least two medicament delivery member cover locks disposed in a circumferential direction of the medicament delivery member cover and flexibly extendable in a radial outward direction with respect to the longitudinal axis, wherein in the extended position and the retracted position, the flexible medicament delivery member cover locks are configured to abut an inner circumferential surface of the housing in a pre-tensioned state, and wherein in the final position after a medicament delivery, the medicament delivery member cover locks are configured to flex and extend in the radial outward direction. The housing comprises at least two relief pockets, wherein each relief pocket is provided in the outer circumferential surface of the housing at a certain distance from a proximal end of the housing. Each relief pocket is configured to receive at least a portion of the respective medicament delivery member cover lock.

Various embodiments may preferably implement the following features.

Preferably, in the final position, a distal end of each of the medicament delivery member cover locks extends further in the radial outward direction than an inner circumferential surface of the housing.

Preferably, the medicament delivery member cover locks are formed as snaps or latches.

Preferably, a width of the relief pocket substantially corresponds to a width of the medicament delivery member cover lock.

Preferably, the relief pocket is formed as a cut-out.

Preferably, the relief pocket is formed as a recess.

Preferably, a housing pocket is provided at the proximal end of the housing and is formed as a chamber extending from an outer circumferential surface of the housing in the radial outward direction, wherein the relief pocket is provided in the housing pocket.

Preferably, the medicament delivery member cover lock comprises a locking extension protruding from the distal end of the medicament delivery member cover lock in a perpendicular direction, wherein the locking extension is configured to be received by the relief pocket.

Preferably, the relief pocket is a slot.

The invention is specified by the independent claim. Preferred embodiments are defined in the dependent claims. In the following description, although numerous features may be designated as optional, it is nevertheless acknowledged that all features comprised in the independent claims are not to be read as optional.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which during use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which during use of the medicament delivery device is/are located closest to the dose delivery site.

Further, the terms “longitudinal”, “longitudinally”, “axially” and “axial” refer to a direction extending from the proximal end to the distal end and along the device or components thereof, typically in the direction of the longest extension of the device and/or component.

Similarly, the terms “transverse”, “transversal” and “transversally” refer to a direction generally perpendicular to the longitudinal direction.

Further, the terms “circumference”, “circumferential”, or “circumferentially” refer to a circumference or a circumferential direction relative to an axis, typically a longitudinal axis extending in the direction of the longest extension of the device and/or component. Similarly, “radial” or “radially” refer to a direction extending radially relative to the axis, and “rotation”, “rotational” and “rotationally” refer to rotation relative to the axis.

When a component is said to move proximally, distally, axially in a proximal direction, axially in a distal direction or equivalent terms, the movement is relative to the housing of the injection device, unless mentioned otherwise.

Generally, all terms used in the claims are to be interpreted according to their ordinary meaning in the technical field, unless explicitly defined otherwise herein. All references to a/an/the element, apparatus, member, component, means, etc. are to be interpreted openly as referring to at least one instance of the element, apparatus, member component, means, etc., unless explicitly stated otherwise.

### BRIEF DESCRIPTION OF THE DRAWINGS

Embodiments of the present disclosure will now be described by way of example only and with reference to the following accompanying drawings.

FIG. 1A shows a medicament delivery member cover according to an example of the present disclosure.

FIG. 1B shows the medicament delivery member cover of FIG. 1A and a housing according to an example of the present disclosure in a locked state.

FIG. 2 shows a partial view of a medicament delivery device comprising the elements of FIGS. 1A and 1B in a locked state.

FIG. 3A shows a medicament delivery member cover according to an example of the present disclosure.

FIG. 3B shows a housing according to an example of the present disclosure in a locked state.

FIG. 3C shows the medicament delivery member cover of FIG. 3A and the housing of FIG. 3B in a locked state.

FIGS. 4A and 4B show partial views of a medicament delivery device comprising the elements of FIGS. 3A to 3C in a locked state.

FIG. 5A shows a medicament delivery member cover according to an example of the present disclosure.

FIG. 5B shows the medicament delivery member cover of FIG. 5A and a housing according to an example of the present disclosure in a locked state.

FIGS. 6A and 6B show partial views of a medicament delivery device comprising the elements of FIGS. 5A and 5B in a locked state.

FIG. 7 shows a sectional view of a medicament delivery device according to FIGS. 3A to 4B.

FIGS. 8A and 8B show partial views of a medicament delivery member cover and a housing according to a non-limiting embodiment of the invention, in a locked state.

FIG. 8C shows a cross-sectional view of the embodiment of FIGS. 8A and 8B.

FIG. 9A shows a partial perspective view of a medicament delivery member cover and a housing according to a non-limiting embodiment of the invention, in a locked state.

FIGS. 9B and 9C are cross-sectional views of the embodiment of FIG. 9A.

FIG. 10A shows a perspective cross-sectional view of a medicament delivery member cover, a housing and a cap according to an example of the present disclosure in an assembled state.

FIG. 10B Shows a Cross-sectional View of the Embodiment of FIG. 10A in a locked state.

FIG. 11A shows a perspective cross-sectional view of a medicament delivery member cover, a housing and a cap according to a non-limiting embodiment of the invention, in an assembled state.

Fig. 11B Shows a Perspective View of the Embodiment of FIG. 11A, in the assembled state.

FIG. 11C shows a perspective view of the embodiment of FIG. 11A, in a locked state.

FIG. 11D shows a cross-sectional view of the embodiment of FIG. 11A, in the locked state.

### DETAILED DESCRIPTION

The lockout mechanism according to the present invention can be employed for various medicament delivery devices. Thus, only the parts relevant for carrying out the disclosure will be described in detail.

In the figures, elements not essential for carrying out the invention have been omitted. In particular, not all figures show a complete outer housing, biasing members, a medicament container, cap, etc. in order not to render the depictions overly complicated and not to blur the scope of the disclosure.

In the following, the assembled state refers to a state in which the device is delivered to the patient, i.e., before activation and medicament delivery. In the assembled state, the plunger rod is in a locked position and not able to move in the proximal direction. In the activation state, the medicament delivery member cover is moved in the distal direction along the longitudinal axis from an extended position to a retracted position. In the activated state, the plunger rod is free to move in the proximal direction for medicament delivery. The plunger rod may thus be displaceable along the axial direction.

The medicament delivery member cover can be moved from the extended to the retracted position for activation of the medicament delivery sequence. Therefore, the medicament delivery member cover can be displaced in the axial direction. In the extended position, the medicament delivery member cover extends in a proximal direction from the housing. Thereby, a medicament delivery member, e.g., a needle, may be covered by the medicament delivery member cover. By pushing the medicament delivery member cover in the distal direction, e.g., by pushing the device onto a medicament delivery site, the medicament delivery member cover is moved to the retracted position for activating medicament delivery. After delivery, the medicament delivery member cover may be moved to a final position (locked position) to cover the medicament delivery member. The final position of the medicament delivery member cover may be further proximal than the extended position. The medicament delivery member cover may also be locked in the final position after medicament delivery to prevent injuries of the patient.

The medicament delivery device may comprise a medicament delivery member cover biasing member configured to exert a force on the medicament delivery member cover 1 in the proximal direction.

In the following, the term engagement encompasses any kind of interaction between the named features such as abutment, clamping, locking, biasing, flexing, etc.

The embodiments described below are examples of the present disclosure and may be combined unless indicated otherwise.

The present disclosure relates to a medicament delivery device comprising, inter alia, a housing 3 comprising a longitudinal axis and a medicament delivery member cover 1 axially moveable within the housing 3 between an extended position, a retracted position and a final position. The medicament delivery member cover 1 comprises at least two flexible medicament delivery member cover locks 12 flexible extendable in a radial outward direction with respect to the longitudinal axis. The medicament delivery member cover locks 12 are disposed in a circumferential direction of the medicament delivery member cover 1. In the extended position and the retracted position, the flexible medicament delivery member cover locks 12 are configured to abut an inner circumferential surface of the housing 3 in a pre-tensioned state. In the final position after a medicament delivery, the medicament delivery member cover locks 12 are configured to flex in the radial outward direction. Further, in the final position, a distal end of each of the medicament delivery member cover locks 12 extends further in the radial outward direction than an inner circumferential surface of the housing 3.

Moreover, in the final position, the medicament delivery member cover locks 12 may abut a surface 31, 33 of the housing 3 that is proximally directed towards a medicament delivery site when the medicament delivery member cover 1 is pushed in the distal direction.

The abutment of the medicament delivery member cover locks 12 on the proximally directed surface 31, 33 of the housing 3 may be configured to block movement of the medicament delivery member 1 in a distal direction away from the medicament delivery site with respect to the housing 3.

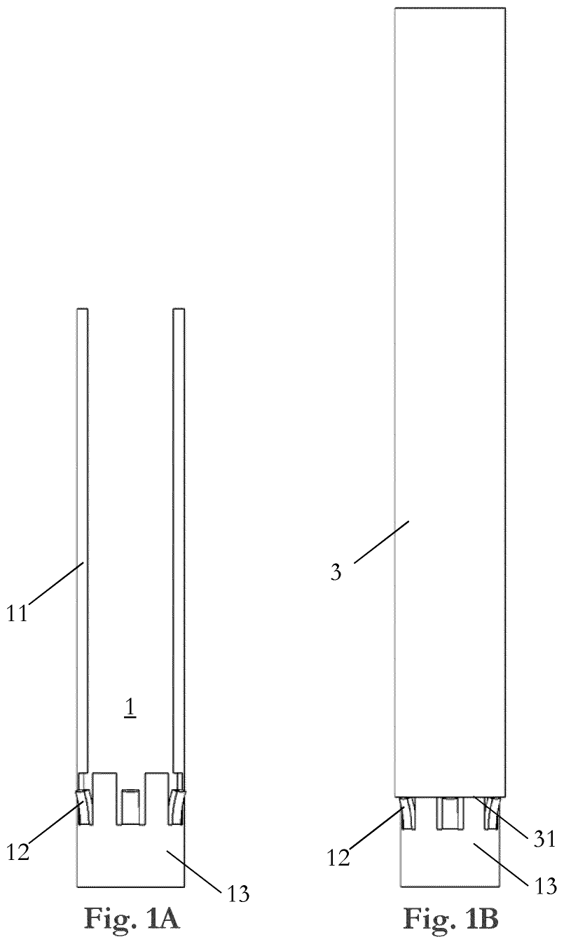

FIG. 1A shows a medicament delivery member cover 1 according to an embodiment. The medicament delivery member cover 1 (needle cover) comprises medicament delivery member cover locks 12 (needle cover locks) extending in a radial outward direction with respect to the longitudinal axis. The medicament delivery member cover locks 12 may be pre-tensioned/biased/compressed in the radial inward direction. In particular, the medicament delivery member cover locks 12 may be flexible in the radial direction. FIG. 1A shows the medicament delivery member cover locks 12 in their relaxed state, i.e., at the most outward radial extension. In other words, in the pre-tensioned state, the distal ends of the medicament delivery member locks 12 are located further inward in the radial direction than in the relaxed state. In the pre-tensioned/biased/compressed state, the medicament delivery member cover locks 12 may provide a force in the radial outward direction due to their compression.

The medicament delivery member cover locks 12 may be formed by snaps, flaps, arms, or similar elements.

The medicament delivery member cover 1 may comprise a substantially cylindrical portion 13 at the proximal end of the medicament delivery member cover 1. The medicament delivery member cover locks 12 may be provided towards or at a distal end of the cylindrical portion 13. As shown in the embodiment of FIG. 1A, the medicament delivery member cover locks 12 extend in distal direction but do not extend to the distal end of the cylindrical portion. The axial position of the medicament delivery member cover locks 12 between the proximal and distal end of the cylindrical portion 13 may be freely chosen without affecting the functionality per se.

The medicament delivery member cover 1 may comprise at least two arms 11 (for example three or four) extending in the distal direction. In particular, the arms 11 may extend from the substantially cylindrical portion 13 in the distal direction.

According to this embodiment, a plurality of medicament delivery member cover locks 12, in particular more than two (for example three or four or, as shown in the figures, six), are provided to intentionally complicate tampering with the device and moving the medicament delivery member cover 1 in the distal direction after medicament delivery.

The medicament delivery member cover locks 12 may particularly be equally distributed on the medicament delivery member cover 1 in a circumferential direction. That is, the medicament delivery member cover locks 12 may be provided in a constant angular distance with respect to each other. However, an irregular arrangement of the medicament delivery member cover locks 12 in the circumferential direction is also possible without compromising the functionality.

Distal ends of each of the medicament delivery member cover locks 12 may be at a same axial position to ensure, that all medicament delivery member cover locks 12 simultaneously abut the proximally directed surface of the housing 3.

Alternatively, two or more medicament delivery member cover locks 12 may be distributed around the circumference of the medicament delivery member cover 1 at different axial positions. Also, the medicament delivery member cover locks 12 may have more than one lock position, e.g. by implementing a zip tie functionality or a step-like shape. Thereby, tolerance sensitivity could be improved, such that lock out may be achieved at an absolutely fully extended medicament delivery member cover. In other words, not all medicament delivery member cover locks 12 need to engage with the housing, but at least some of the medicament delivery member cover locks 12 may be engaged without unnecessary overshooting. In this manner, a tighter lockout position, and better prevention of the needle being exposed may be achieved compared to a looser medicament delivery member cover lock 12.

During assembly, the medicament delivery member cover 1 is inserted into the housing 3 such that the medicament delivery member cover locks 12 are flexed inwards and abut an inner circumferential surface of the housing 3. The housing 3 may be substantially cylindrical. That is, in the extended position and the retracted position, the medicament delivery member cover locks 12 abut, i.e. rest at, and slide along the inner circumferential surface of the housing 3. The distal end of the medicament delivery member cover locks 12 are therefore not located at a position further proximal than the proximal end surface 31 of the housing 3 when the medicament delivery member cover 1 is in the extended position or in the retracted position. The medicament delivery member cover locks 12 are in a compressed state and, as indicated above, pre-tensioned in a radial inward direction.

When, after medicament delivery, the medicament delivery member cover 1 is pushed in the proximal direction, e.g., by removing the device from a medicament delivery site and by exerting a proximally directed force (for example by a spring) on the medicament delivery member cover 1, the medicament delivery member cover locks 12 can flex outwards as soon as they pass the proximal end surface 31 (proximally directed surface) of the housing 3. The medicament delivery member cover locks 12 thus abut the proximal end surface 31 and prevent the medicament delivery member cover 1 from being pushed in the distal direction. The medicament delivery member cover 1 is blocked in the distal direction.

FIG. 1B shows the device in the locked state, i.e., with the medicament delivery member cover 1 in the final position (locked position) and with the medicament delivery member cover locks 12 being in the most radially outward (i.e., relaxed) position and thus abutting the proximal end surface 31.

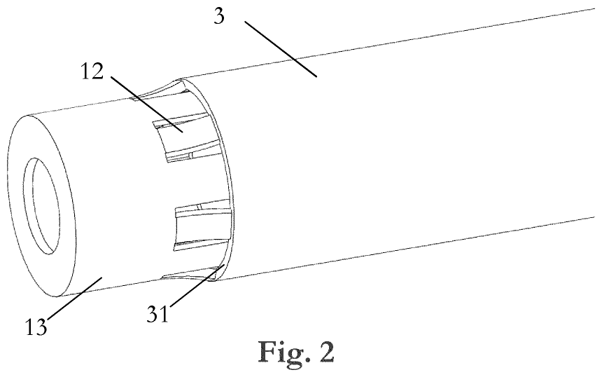

FIG. 2 Is a Perspective and Enlarged View of FIG. 1B.

In the above figures, the medicament delivery member cover locks 12 are depicted as directly contacting the proximal end surface 31 in the final position (locked position). However, the final position may also refer to a position, in which the locks 12 are located further proximally than the proximal end surface 31 and only abut the proximal end surface 31 in case the medicament delivery member cover 1 is pushed distally a certain distance.

In other words, in the final position, a distal end or at least the distal end of the medicament delivery member cover locks 12 may extend further in the radial outward direction than an inner circumferential surface of the housing 3 as shown in FIGS. 1A to 6B.

It may be advantageous, however, that the medicament delivery member is covered by the medicament delivery member cover at all times after medicament delivery, that is, after removal of the device from the injection site.

The medicament delivery device may comprise a medicament delivery member (not shown), particularly a needle. The medicament delivery member cover 1 may be configured to cover the medicament delivery member in the extended position and the final position. In the retracted position of the medicament delivery member cover 1, the medicament delivery member may extend further proximally than the proximal end of the housing 3 and the medicament delivery member cover 1 for piercing a patient's skin.

For a user, it will turn out to be difficult to compress more than two medicament delivery member cover locks 12/snaps at once. Moreover, the provision of more snaps provides an indication to the user that it is not intended to move the medicament delivery member cover 1 after medicament delivery.

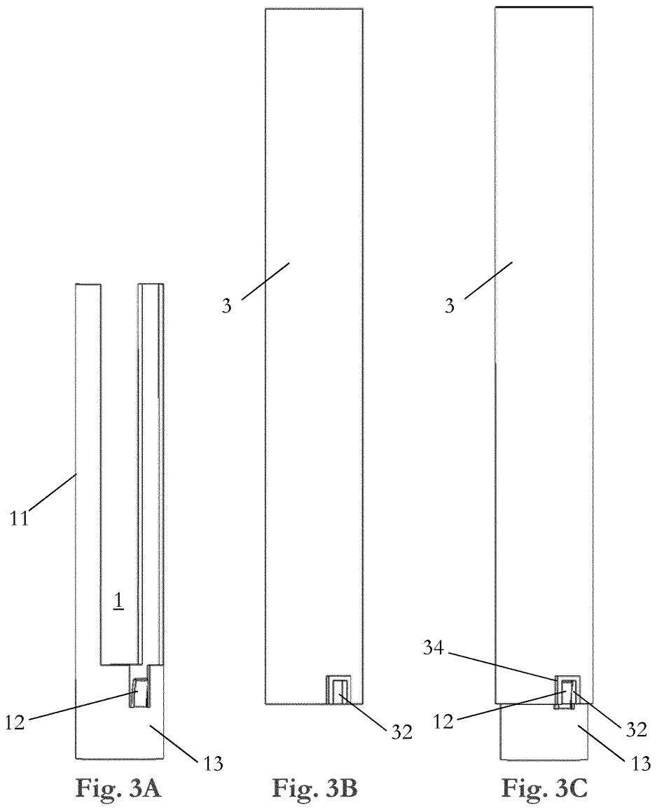

In the following, embodiments according to FIGS. 3A to 6B are described. Unless explicitly indicated otherwise, the above description equally applies to the following embodiments. The detailed description of equal or similar features is thus omitted.

FIG. 3A shows a medicament delivery member cover 1 according to an embodiment. The medicament delivery member cover 1 comprises at least two medicament delivery member cover locks 12. Said locks 12 may be formed as snaps or latches or flexible arms. The medicament delivery member cover may further comprise a cylindrical portion 13 on which the medicament delivery member cover locks 12 are provided and may comprise at least two medicament delivery member cover arms 11 as described above.

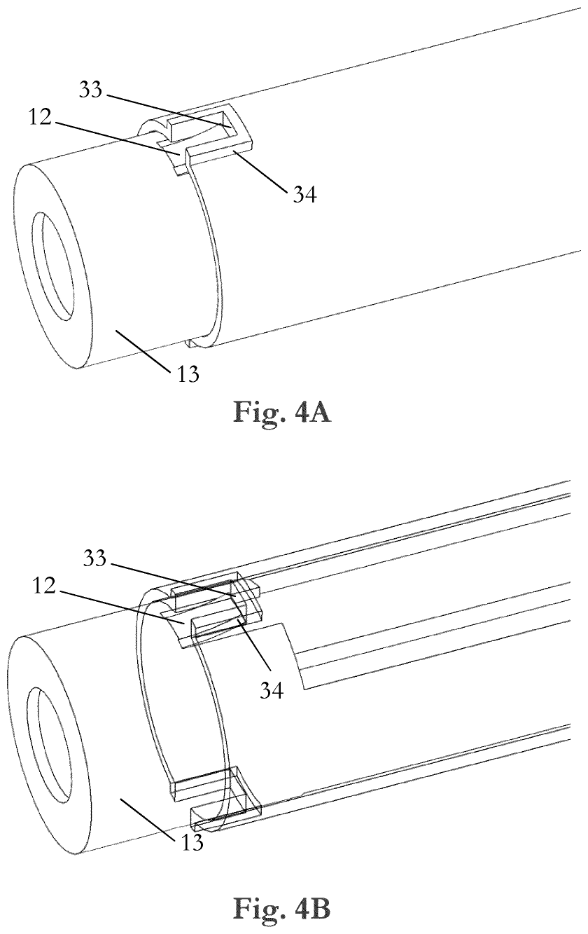

FIG. 3B shows a housing 3 according to an embodiment. The housing 3 comprises at least two pockets 32. The number of pockets 32 may correspond to the number of medicament delivery member cover locks 12. The circumferential distance of the pockets 32 corresponds to the circumferential distance of the medicament delivery member cover locks 12. The pockets 32 may be formed as an axial recess extending from a proximal end of the housing 3 in the distal direction. In particular, the pockets 32 may be formed as a cut-out. The proximally directed surface may respectively be formed by a pocket abutment surface 33 of a distal end of the pocket 32 (see FIGS. 4A and 4B). The pockets 32 may extend in a radially outward direction.

FIG. 3C shows the medicament delivery member cover 1 of FIG. 3A and the housing 3 according to FIG. 3B the locked state.

FIG. 4A is a partial perspective view of FIG. 3C. As can be seen from FIG. 4A, the pocket abutment surface 33 forming the proximally directed surface on which the medicament delivery member cover locks 12 abut, are formed by the distal end surface of the pocket 32.

The pocket 32 may further comprise an elevated side wall or rim 34. The elevated side wall 34 may extend along the lateral sides of the pocket 32, i.e., along the sides extending in the axial direction, and along the pocket abutment surface 33 at the distal end of the pocket 32 which extends in the circumferential direction. The elevated side wall 34 may extend in a radial outward direction, i.e., protrude from an outer circumferential surface of the housing 3.

A width of the pockets 31 may substantially correspond to a width of the medicament delivery member cover locks 12.

FIG. 4B shows the view of FIG. 4A, wherein the housing 3 is transparently displayed.

As described above, the medicament delivery member cover locks 12 may be formed as snaps or latches and pre-tensioned such that they are biased in the radially inward direction. In the extended and the retracted position, the locks 12 abut and slide along the inner circumferential surface of the housing 3. When the medicament delivery member cover 1 is moved to the final position (locked position), the distal end of the medicament delivery member locks 12 pass the distal end of the respective pockets 32 (i.e., the proximally directed surface/pocket abutment surface 33). Consequently, the locks 12 flex outwardly and abut the pocket abutment surface 33 to block movement of the medicament delivery member cover 11 in the distal direction. As explained above, the final position (locked position) may also refer to a position in which the locks 12 are positioned at a certain distance from the pocket abutment surface 33. However, when the medicament delivery member cover 1 is pushed in the distal direction, after said certain distance the medicament delivery member cover locks abut the pocket abutment surface 33 and thus block further movement of the medicament delivery member cover 1 in the distal direction.

By locking the medicament delivery member cover locks 12 in the respective pockets 32, the access to the lock 12 is obstructed and they are difficult to reach with a finger to press them radially inside. By additionally providing the elevated side wall 34, access to the locks 12 and thus tampering with the lockout mechanism may be further obstructed.

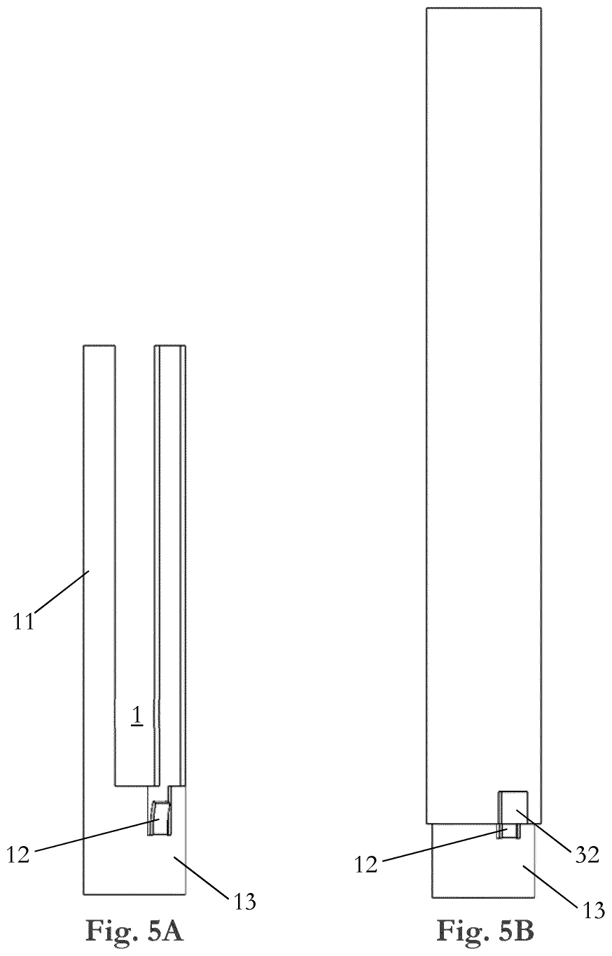

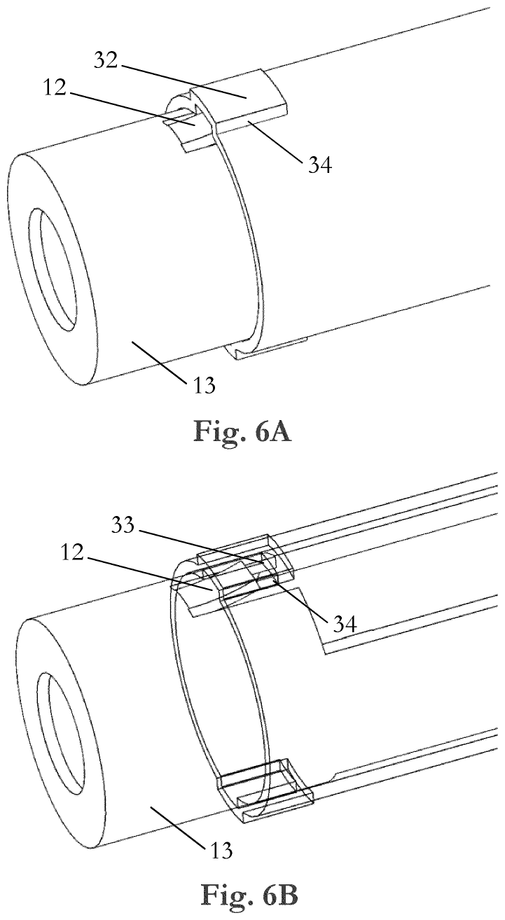

FIGS. 5A to 6B show an embodiment closely related to the embodiment of FIGS. 3A to 4B. As indicated above, the above description of FIGS. 1A to 4B equally applies, unless explicitly stated otherwise.

FIG. 5A shows a medicament delivery member cover 1 substantially corresponding to the medicament delivery member cover 1 of FIG. 3A.

The housing shown in FIG. 5B substantially corresponds to the housing of FIG. 3C, wherein the pocket 32 is covered. In particular, an outer circumferential surface of the pocket 32 may be covered such that the lock 12 provided therein in the final position (locked position) may not be reached by a user. In other words, the pocket 32 may be covered in the radial outward direction. The pocket 32 is formed as a closed pocket. As in the previous embodiment, an elevated side wall 34 may be provided. The elevated side wall 34 may protrude in the radial outward direction from an outer circumferential surface of the housing 3 and forms a pocket cover as shown in FIGS. 5B to 6B. In other words, the elevated side wall 34 is circumferentially concentric to the circumferential outer surface of the housing 3.

FIG. 6A is a partial perspective view of FIG. 5B and shows the medicament delivery member cover 1 and the housing 3 in the final state (locked state).

FIG. 6B shows the same view as FIG. 6A, wherein the housing 3 is transparently displayed. As can be seen from FIG. 6B, the lock 12, in case the medicament delivery member cover 1 is pushed distally, abuts the proximally directed abutment surface 33 of the pocket 32, thus blocking axial movement of the medicament delivery member cover 1 beyond said abutment.

By additionally covering the pockets 32 with the elevated side wall 34, access to the locks 12 may be further obstructed and safety of the device may thus be improved.

Moreover, the embodiments of FIGS. 3 to 6 may facilitate assembly of the device with respect to the embodiment of FIGS. 1 and 2, since the medicament delivery member cover locks 12 (snaps/latches) have to be compressed during assembly to fit within the housing 3. This process might be easier having less locks 12 to be compressed.

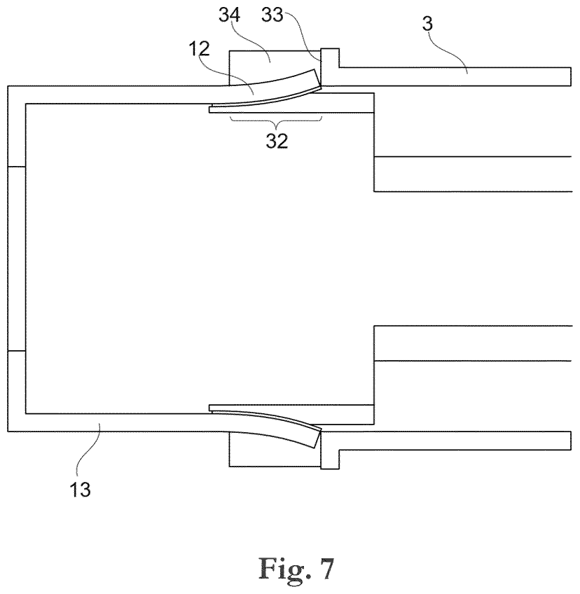

FIG. 7 is a sectional view of the device according to FIGS. 3A to 4B along the longitudinal axis. This sectional view is exemplary for all embodiments disclosed herein and is merely used to illustrate the functional principle of the present disclosure.

As can be seen from FIG. 7, the medicament delivery member cover locks 12 are bent or flexed outward in the radial direction and abut the abutment surface 33 of the pocket 32.

Further to the above embodiments, it might be advantageous to design the medicament delivery member cover locks 12 (snaps, latches) such that independent of the storage period and potential changes in material properties, the correct functioning, i.e., locking of the device after medicament delivery, is ensured. The below description is fully compatible with the above embodiments and may be combined unless indicated otherwise. Therefore, a detailed description of some of the features will be omitted to avoid redundancies.

In particular, the medicament delivery member cover locks 12 might comprise a maximum extension D in the radial outward direction after manufacture. The maximum extension D may be defined as the radial extension after manufacture or prior to assembly of the device. Due to creep, fatigue or the like, the maximum extension D might not always be reached, in particular since according to the present disclosure, the medicament delivery member cover locks 12 are stored in a compressed (pre-tensioned) state. That is, the medicament delivery member cover locks 12 may be stored abutting the inner circumferential surface of the housing 3 as explained above (extended position and retracted position). Depending on the storage conditions, such as humidity, temperature, etc. as well as the storage time, the material may be subject to creep and the intended pretension or tension may not be fully achieved. In the worst case, the medicament delivery member cover locks 12 do not extend beyond the inner circumferential surface of the housing 3 such that no abutment takes place and the medicament delivery member cover 1 is not locked in the final position.

In the final position, a distal end of each of the medicament delivery member cover locks 12 extends further in the radial outward direction than an inner circumferential surface of the housing 3 to ensure locking of the medicament delivery member cover 1 after medicament delivery.

To ensure that the medicament delivery member cover locks 12 achieve said extension at least up to the inner circumferential surface of the housing 3, they may be configured to maintain at least 50% of the maximum extension D during storage in the extended position. In other words, since the amount of creep is dependent on the storage conditions such as time, temperature, humidity etc., the elements of the medicament delivery device are designed such that sufficient engagement of the medicament delivery member cover locks 12 and the corresponding abutment surface is ensured if at least 50% of the original pretension is maintained. Thus, the features disclosed herein accommodate for retaining the functionality given a certain percentage of pretension loss during storage.

In other words, the medicament delivery member cover locks 12 may be configured to maintain during storage in the extended position pretension to obtain/assume/achieve at least 50% of the maximum extension D in the final position.

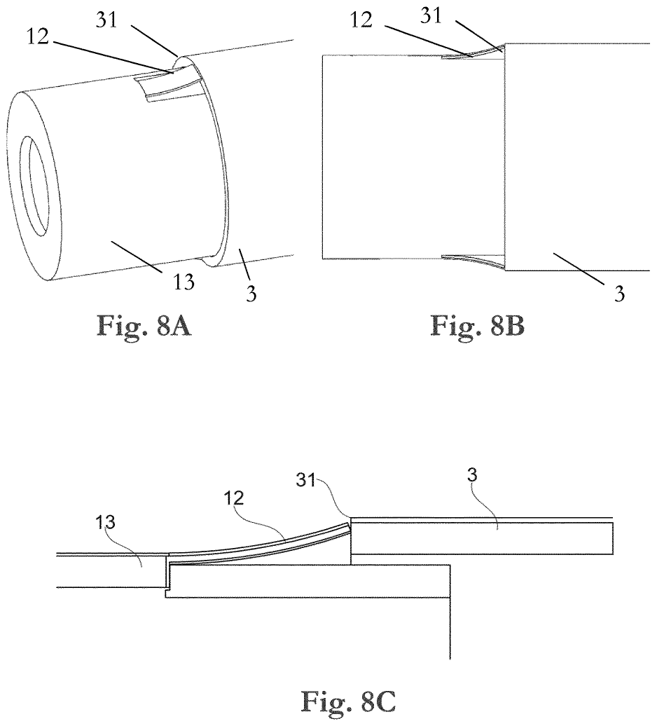

This may be achieved by forming the medicament delivery member cover locks 12 of a different material. As shown in FIGS. 8A to 8C, the medicament delivery member cover locks 12 may be formed of metal. In general, metal is less subject to creep over long storage periods and may thus substantially maintain its original flexibility and pre-tension.

Moreover, maintaining the pretension for locking the medicament delivery member cover 1 may be achieved by manufacturing (e.g., moulding) the medicament delivery member cover locks 12 such that they extend further than the intended lockout. This allows the medicament delivery member cover locks 12 to still function after they lose some of their pretension to creep.

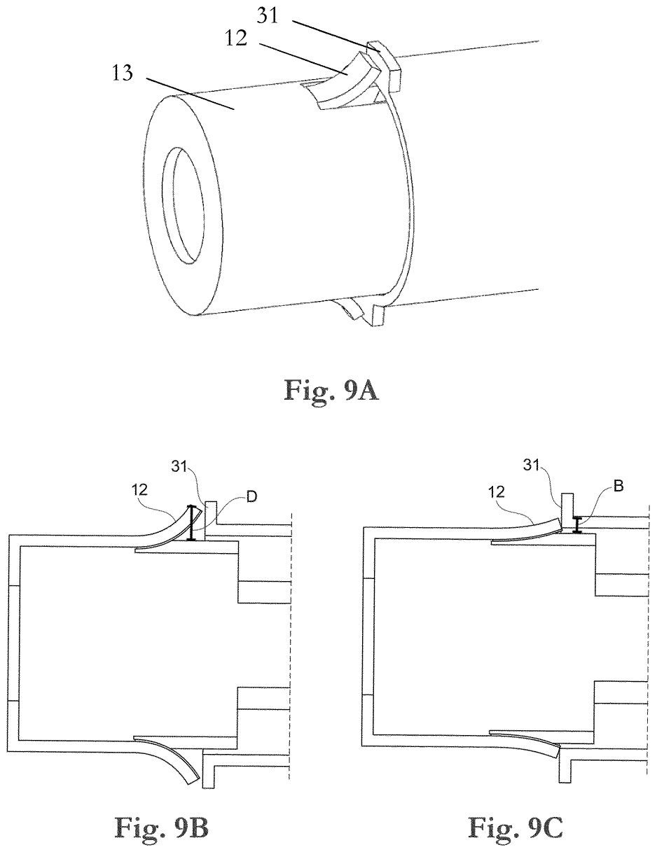

This is shown in FIG. 9A which is a perspective view of a medicament delivery member cover 1 and a housing 3 having a proximal abutment surface 31, in this case an elevated proximal abutment surface 31 extending in a radial outward direction, according to an embodiment. FIG. 9A shows the medicament delivery member cover locks 12 in a relaxed state after manufacture. The medicament delivery member cover 1 is depicted in the final position, i.e., the device is in the locked state. Due to the abutment of the medicament delivery member cover locks 12 on the proximal abutment surface 31, the medicament delivery member cover 1 cannot be pushed in the distal direction inside the housing. The medicament delivery member cover locks 12 reach their radial maximum extension D (maximum distance).

FIGS. 9B and 9C are cross-sectional views of the device according to FIG. 9A. In FIG. 9B, the medicament delivery member cover locks 12 reach the maximum radial extension D (relaxed state/locked state), while in FIG. 9C, the medicament delivery member cover locks 12 (also in the relaxed state/locked state) have lost some of the original material properties and thus do not extend as far in the radial direction as originally after manufacture.

As can be seen from FIG. 9B, the maximum extension D may be measured as a radial distance between an outer circumferential surface of the medicament delivery member cover 1 and the distal end (or distally outermost edge) of the respective medicament delivery member cover lock 12.

The maximum extension D may be at least 150% of a radial blocking distance B (see FIG. 9C) between the outer circumferential surface of the medicament delivery member cover 1 and the outer circumferential surface of the housing 3, preferably 200% or more of the blocking distance B. In an exemplary embodiment, the medicament delivery member cover locks 12 may have a maximum extension D of 2 mm in order to maintain at least 1 mm of radial extension after storage.

The blocking distance B has been defined as the distance between the outer circumferential surface of the medicament delivery member cover 1 and the outer circumferential surface of the housing 3 (FIG. 9C), but may also be smaller as long as a blocking of movement of the medicament delivery member cover 1 in the distal direction is ensured.

The delivery member cover locks 12 may also be described by a deflection distance and the blocking distance. The deflection distance denotes the extension during storage, i.e., the distance between the outer circumferential surface of the medicament delivery member cover 1 and the inner circumferential surface of the housing 3 (the deflection distance is not indicated in the figures). After medicament delivery, the medicament delivery member cover locks 12 may at least extend up to the blocking distance B.

In the latter case, the blocking distance B may be at least 150% of the deflection distance, preferably 200% of the deflection distance or more.

In any case, the medicament delivery member cover locks 12 may be configured to maintain during storage pretension to achieve at least the blocking distance B in the final position.

According to the above properties, it can be ensured that enough tension in the radial direction is left also after long storage periods to ensure locking of the device after use.

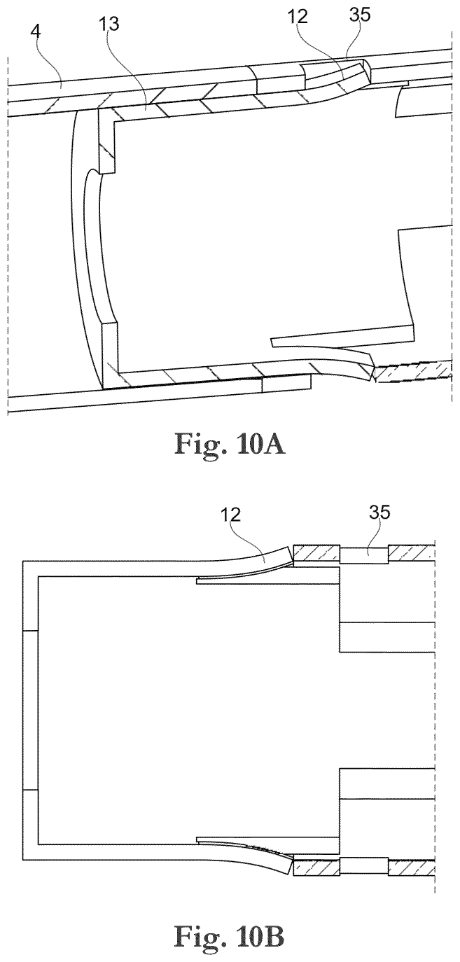

As shown in FIG. 10A, the housing 3 may also comprises at least two relief pockets 35 (or in general one relief pocket per medicament delivery member cover lock 12). Each relief pocket 35 may be provided in the outer circumferential surface of the housing 3 at a certain distance from a proximal end of the housing 3. FIG. 10A shows the device in the assembled state, i.e. as delivered to the user, with the cap 4 attached and the medicament delivery member cover 1 in the extended position.

Each relief pocket 35 may be configured to receive at least a portion of the respective medicament delivery member cover lock 12. Hence, the medicament delivery member cover locks 12 can be stored in a relaxed state rather than a compressed state. The medicament delivery member cover locks 12 may thus maintain their original pretension since less stress is exerted thereon.

FIG. 10B shows a cross-sectional view of the device according to FIG. 10A after medicament delivery, i.e., with the medicament delivery member cover 1 in the final position (locked state). As can be seen from the figure, the medicament delivery member cover locks 12 still extend outward to such an extent that locking of the device is ensured.

A width of the relief pocket 35 may substantially correspond to a width of the medicament delivery member cover lock 12. The relief pocket 35 may be formed as a cut-out or a recess.

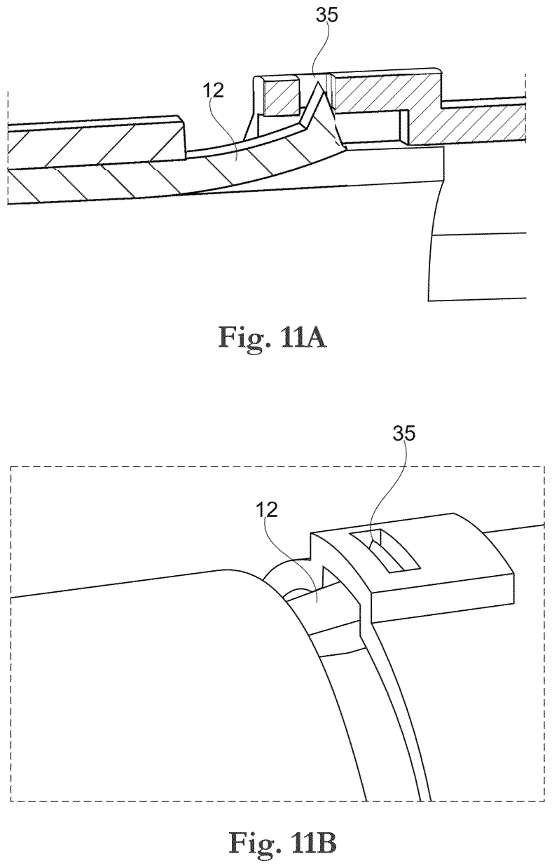

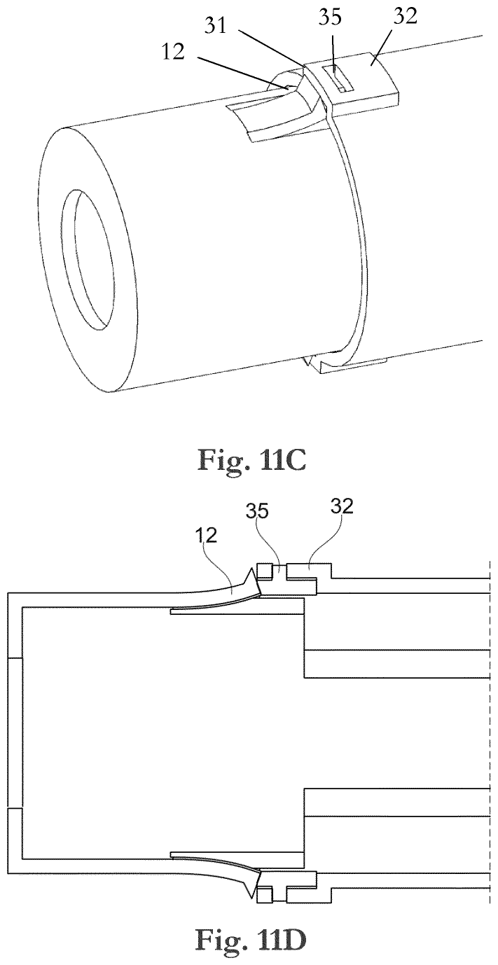

As shown in FIGS. 11A to 11D, a housing pocket 32 as described above in conjunction with FIGS. 5A to 6B may be provided on the housing 3, in particular at the proximal end of the housing 3. The housing pocket 32 may be formed as a chamber extending from an outer circumferential surface of the housing 3 in the radial outward direction. The relief pocket 35 may be provided in the housing pocket 32.

For example, the relief pocket 35 may be formed as a slot as depicted in FIG. 11A showing a cross-sectional view of a medicament delivery device in an assembled state (i.e., as delivered to the user) with the cap attached and the medicament delivery member cover locks 12 inside the relief pocket 35. The medicament delivery member cover locks 12 are thus in a relaxed state.

FIG. 11B Is a Perspective View of FIG. 11A.

The medicament delivery member cover locks 12 may comprise a locking extension protruding from the distal end of the medicament delivery member cover lock 12 in a perpendicular direction. The locking extension may be configured to be received by the relief pocket 35 as shown in FIGS. 11A and 11B. The relief pocket 35 may have a chamfer forcing the medicament delivery member cover lock 12 to flex inside when the medicament delivery member cover 1 is pushed in the distal direction for device activation.

FIGS. 11C and 11D show the device of FIGS. 11A and 11B with the medicament delivery member cover 1 in the final position (locked state) and the medicament delivery member cover locks 12 abutting the proximal end surface 31 of the housing 3. In this case, the abutment surface or proximal end surface 31 is formed by a proximal surface of the housing pocket 32.

Due to the storage in the relief pocket 35, the medicament delivery member cover locks 12 have maintained the original tension and locking of the device is ensured also after long storage periods.

According to the present disclosure, handling of the medicament delivery device after medicament delivery may be facilitated while preventing unintentionally reaching the needle and thus avoiding injuries of a user. In particular, it may be avoided that the lockout after use of the medicament delivery does not work correctly due to creep or the like in the locking snaps.

The delivery devices described herein can be used for the treatment and/or prophylaxis of one or more of many different types of disorders.

Exemplary disorders include, but are not limited to: rheumatoid arthritis, inflammatory bowel diseases (e.g. Crohn's disease and ulcerative colitis), hypercholesterolaemia and/or dyslipidemia, cardiovascular disease, diabetes (e.g. type 1 or 2 diabetes), psoriasis, psoriatic arthritis, spondyloarthritis, hidradenitis suppurativa, Sjögren's syndrome, migraine, cluster headache, multiple sclerosis, neuromyelitis optica spectrum disorder, anaemia, thalassemia, paroxysmal nocturnal hemoglobinuria, hemolytic anaemia, hereditary angioedema, systemic lupus erythematosus, lupus nephritis, myasthenia gravis, Behçet's disease, hemophagocytic lymphohistiocytosis, atopic dermatitis, retinal diseases (e.g., age-related macular degeneration, diabetic macular edema), uveitis, infectious diseases, bone diseases (e.g., osteoporosis, osteopenia), asthma, chronic obstructive pulmonary disease, thyroid eye disease, nasal polyps, transplant, acute hypoglycaemia, obesity, anaphylaxis, allergies, sickle cell disease, Alzheimer's disease, Parkinson's disease, dementia with Lewy bodies, systemic infusion reactions, immunoglobulin E (IgE)-mediated hypersensitivity reactions, cytokine release syndrome, immune deficiencies (e.g., primary immunodeficiency, chronic inflammatory demyelinating polyneuropathy), enzyme deficiencies (e.g., Pompe disease, Fabry disease, Gaucher disease), growth factor deficiencies, hormone deficiencies, coagulation disorders (e.g., hemophilia, von Willebrand disease, Factor V Leiden), and cancer.

Exemplary types of drugs that could be included in the delivery devices described herein include, but are not limited to, small molecules, hormones, cytokines, blood products, enzymes, vaccines, anticoagulants, immunosuppressants, antibodies, antibody-drug conjugates, neutralizing antibodies, reversal agents, radioligand therapies, radioisotopes and/or nuclear medicines, diagnostic agents, bispecific antibodies, proteins, fusion proteins, peptibodies, polypeptides, pegylated proteins, protein fragments, nucleotides, protein analogues, protein variants, protein precursors, protein derivatives, chimeric antigen receptor T cell therapies, cell or gene therapies, oncolytic viruses, or immunotherapies.

Exemplary drugs that could be included in the delivery devices described herein include, but are not limited to, immuno-oncology or bio-oncology medications such as immune checkpoints, cytokines, chemokines, clusters of differentiation, interleukins, integrins, growth factors, coagulation factors, enzymes, enzyme inhibitors, retinoids, steroids, signaling proteins, pro-apoptotic proteins, anti-apoptotic proteins, T-cell receptors, B-cell receptors, or costimulatory proteins.

Exemplary drugs that could be included in the delivery devices described herein include, but are not limited to, those exhibiting a proposed mechanism of action, such as human epidermal growth factor receptor 2 (HER-2) receptor modulators, interleukin (IL) modulators, interferon (IFN) modulators, complement modulators, glucagon-like peptide-1 (GLP-1) modulators, glucose-dependent insulinotropic polypeptide (GIP) modulators, cluster of differentiation 38 (CD38) modulators, cluster of differentiation 22 (CD22) modulators, C1 esterase modulators, bradykinin modulators, C-C chemokine receptor type 4 (CCR4) modulators, vascular endothelial growth factor (VEGF) modulators, B-cell activating factor (BAFF), P-selectin modulators, neonatal Fc receptor (FcRn) modulators, calcitonin gene-related peptide (CGRP) modulators, epidermal growth factor receptor (EGFR) modulators, cluster of differentiation 79B (CD79B) modulators, tumor-associated calcium signal transducer 2 (Trop-2) modulators, cluster of differentiation 52 (CD52) modulators, B-cell maturation antigen (BCMA) modulators, enzyme modulators, platelet-derived growth factor receptor A (PDGFRA) modulators, cluster of differentiation 319 (CD319 or SLAMF7) modulators, programmed cell death protein 1 and programmed death-ligand 1 (PD-1/PD-L1) inhibitors/modulators, B-lymphocyte antigen cluster of differentiation 19 (CD19) inhibitors, B-lymphocyte antigen cluster of differentiation 20 (CD20) modulators, cluster of differentiation 3 (CD3) modulators, cytotoxic T-lymphocyte-associated protein 4 (CTLA-4) inhibitors, T-cell immunoglobulin and mucin-domain containing-3 (TIM-3) modulators, T cell immunoreceptor with Ig and ITIM domains (TIGIT) modulators, V-domain Ig suppressor of T cell activation (VISTA) modulators, indoleamine 2,3-dioxygenase (IDO or INDO) modulators, poliovirus receptor-related immunoglobulin domain-containing protein (PVRIG) modulators, lymphocyte-activation gene 3 (LAG3; also known as cluster of differentiation 223 or CD223) antagonists, cluster of differentiation 276 (CD276 or B7-H3) antigen modulators, cluster of differentiation 47 (CD47) antagonists, cluster of differentiation 30 (CD30) modulators, cluster of differentiation 73 (CD73) modulators, cluster of differentiation 66 (CD66) modulators, cluster of differentiation w137 (CDw137) agonists, cluster of differentiation 158 (CD158) modulators, cluster of differentiation 27 (CD27) modulators, cluster of differentiation 58 (CD58) modulators, cluster of differentiation 80 (CD80) modulators, cluster of differentiation 33 (CD33) modulators, cluster of differentiation 159 (CD159 or NKG2) modulators, glucocorticoid-induced TNFR-related (GITR) protein modulators, Killer Ig-like receptor (KIR) modulators, growth arrest-specific protein 6 (GAS6)/AXL pathway modulators, A proliferation-inducing ligand (APRIL) receptor modulators, human leukocyte antigen (HLA) modulators, epidermal growth factor receptor (EGFR) modulators, B-lymphocyte cell adhesion molecule modulators, cluster of differentiation w123 (CDw123) modulators, Erbb2 tyrosine kinase receptor modulators, endoglin modulators, mucin modulators, mesothelin modulators, hepatitis A virus cellular receptor 2 (HAVCR2) antagonists, cancer-testis antigen (CTA) modulators, tumor necrosis factor receptor superfamily, member 4 (TNFRSF4 or OX40) modulators, adenosine receptor modulators, inducible T cell co-stimulator (ICOS) modulators, cluster of differentiation 40 (CD40) modulators, tumor-infiltrating lymphocytes (TIL) therapies, or T-cell receptor (TCR) therapies.

Exemplary drugs that could be included in the delivery devices described herein include, but are not limited to: etanercept, abatacept, adalimumab, evolocumab, exenatide, secukinumab, erenumab, galcanezumab, fremanezumab-vfrm, alirocumab, methotrexate (amethopterin), tocilizumab, interferon beta-1a, interferon beta-1b, peginterferon beta-1a, sumatriptan, darbepoetin alfa, belimumab, sarilumab, semaglutide, dupilumab, reslizumab, omalizumab, glucagon, epinephrine, naloxone, insulin, amylin, vedolizumab, eculizumab, ravulizumab, crizanlizumab-tmca, certolizumab pegol, satralizumab, denosumab, romosozumab, benralizumab, emicizumab, tildrakizumab, ocrelizumab, ofatumumab, natalizumab, mepolizumab, risankizumab-rzaa, ixekizumab, and immune globulins.

Exemplary drugs that could be included in the delivery devices described herein may also include, but are not limited to, oncology treatments such as ipilimumab, nivolumab, pembrolizumab, atezolizumab, durvalumab, avelumab, cemiplimab, rituximab, trastuzumab, ado-trastuzumab emtansine, fam-trastuzumab deruxtecan-nxki, pertuzumab, transtuzumab-pertuzumab, alemtuzumab, belantamab mafodotin-blmf, bevacizumab, blinatumomab, brentuximab vedotin, cetuximab, daratumumab, elotuzumab, gemtuzumab ozogamicin, 90-Yttrium-ibritumomab tiuxetan, isatuximab, mogamulizumab, moxetumomab pasudotox, obinutuzumab, ofatumumab, olaratumab, panitumumab, polatuzumab vedotin, ramucirumab, sacituzumab govitecan, tafasitamab, or margetuximab.

Exemplary drugs that could be included in the delivery devices described herein include “generic” or biosimilar equivalents of any of the foregoing, and the foregoing molecular names should not be construed as limiting to the “innovator” or “branded” version of each, as in the non-limiting example of innovator medicament adalimumab and biosimilars such as adalimumab-afzb, adalimumab-atto, adalimumab-adbm, and adalimumab-adaz.

Exemplary drugs that could be included in the delivery devices described herein also include, but are not limited to, those used for adjuvant or neoadjuvant chemotherapy, such as an alkylating agent, plant alkaloid, antitumor antibiotic, antimetabolite, or topoisomerase inhibitor, enzyme, retinoid, or corticosteroid. Exemplary chemotherapy drugs include, by way of example but not limitation, 5-fluorouracil, cisplatin, carboplatin, oxaliplatin, doxorubicin, daunorubicin, idarubicin, epirubicin, paclitaxel, docetaxel, cyclophosphamide, ifosfamide, azacitidine, decitabine, bendamustine, bleomycin, bortezomib, busulfan, cabazitaxel, carmustine, cladribine, cytarabine, dacarbazine, etoposide, fludarabine, gemcitabine, irinotecan, leucovorin, melphalan, methotrexate, pemetrexed, mitomycin, mitoxantrone, temsirolimus, topotecan, valrubicin, vincristine, vinblastine, or vinorelbine.

Exemplary drugs that could be included in the delivery devices described herein also include, but are not limited to, analgesics (e.g., acetaminophen), antipyretics, corticosteroids (e.g. hydrocortisone, dexamethasone, or methylprednisolone), antihistamines (e.g., diphenhydramine or famotidine), antiemetics (e.g., ondansetron), antibiotics, antiseptics, anticoagulants, fibrinolytics (e.g., recombinant tissue plasminogen activator [r-TPA]), antithrombolytics, or diluents such as sterile water for injection (SWFI), 0.9% Normal Saline, 0.45% normal saline, 5% dextrose in water, 5% dextrose in 0.45% normal saline, Lactated Ringer's solution, Heparin Lock Flush solution, 100 U/mL Heparin Lock Flush Solution, or 5000 U/mL Heparin Lock Flush Solution.

Pharmaceutical formulations including, but not limited to, any drug described herein are also contemplated for use in the delivery devices described herein, for example pharmaceutical formulations comprising a drug as listed herein (or a pharmaceutically acceptable salt of the drug) and a pharmaceutically acceptable carrier. Such formulations may include one or more other active ingredients (e.g., as a combination of one or more active drugs), or may be the only active ingredient present, and may also include separately administered or co-formulated dispersion enhancers (e.g. an animal-derived, human-derived, or recombinant hyaluronidase enzyme), concentration modifiers or enhancers, stabilizers, buffers, or other excipients.

Exemplary drugs that could be included in the delivery devices described herein include, but are not limited to, a multi-medication treatment regimen such as AC, Dose-Dense AC, TCH, GT, EC, TAC, TC, TCHP, CMF, FOLFOX, mFOLFOX6, mFOLFOX7, FOLFCIS, CapeOx, FLOT, DCF, FOLFIRI, FOLFIRINOX, FOLFOXIRI, IROX, CHOP, R-CHOP, RCHOP-21, Mini-CHOP, Maxi-CHOP, VR-CAP, Dose-Dense CHOP, EPOCH, Dose-Adjusted EPOCH, R-EPOCH, CODOX-M, IVAC, HyperCVAD, R-HyperCVAD, SC-EPOCH-RR, DHAP, ESHAP, GDP, ICE, MINE, CEPP, CDOP, GemOx, CEOP, CEPP, CHOEP, CHP, GCVP, DHAX, CALGB 8811, HIDAC, MOPAD, 7+3, 5+2, 7 +4, MEC, CVP, RBAC500, DHA-Cis, DHA-Ca, DHA-Ox, RCVP, RCEPP, RCEOP, CMV, DDMVAC, GemFLP, ITP, VIDE, VDC, VAI, VDC-IE, MAP, PCV, FCR, FR, PCR, HDMP, OFAR, EMA/CO, EMA/EP, EP/EMA, TP/TE, BEP, TIP, VIP, TPEx, ABVD, BEACOPP, AVD, Mini-BEAM, IGEV, C-MOPP, GCD, GEMOX, CAV, DT-PACE, VTD-PACE, DCEP, ATG, VAC, VeIP, OFF, GTX, CAV, AD, MAID, AIM, VAC-IE, ADOC, or PE.

Various modifications to the embodiments described are possible and will occur to those skilled in the art without departing from the invention which is defined by the following claims.

1-9. (canceled)

10. A medicament delivery device, the medicament delivery device having a proximal end pointing towards a dose delivery site during use of the medicament delivery device, and a distal end opposite the proximal end, the medicament delivery device comprising:
a housing comprising a longitudinal axis; and
a medicament delivery member cover axially moveable within the housing between an extended position, a retracted position and a final position,
the medicament delivery member cover comprising at least two medicament delivery member cover locks disposed in a circumferential direction of the medicament delivery member cover and flexibly extendable in a radial outward direction with respect to the longitudinal axis,
wherein in the extended position and the retracted position, the flexible medicament delivery member cover locks are configured to abut an inner circumferential surface of the housing in a pre-tensioned state,
wherein in the final position after a medicament delivery, the medicament delivery member cover locks are configured to flex and extend in the radial outward direction,
wherein the housing comprises at least two relief pockets, wherein each relief pocket is provided in the outer circumferential surface of the housing at a certain distance from a proximal end of the housing, and
wherein each relief pocket is configured to receive at least a portion of the respective medicament delivery member cover lock.

11. The medicament delivery device according to claim 10, wherein in the final position, a distal end of each of the medicament delivery member cover locks extends further in the radial outward direction than an inner circumferential surface of the housing.

12. The medicament delivery device according to claim 10, wherein the medicament delivery member cover locks are formed as snaps or latches.

13. The medicament delivery device according to claim 10, wherein a width of the relief pocket substantially corresponds to a width of the medicament delivery member cover lock.

14. The medicament delivery device according to claim 10, wherein the relief pocket is formed as a cut-out.

15. The medicament delivery device according to claim 10, wherein the relief pocket is formed as a recess.

16. The medicament delivery device according to claim 10, wherein a housing pocket is provided at the proximal end of the housing and is formed as a chamber extending from an outer circumferential surface of the housing in the radial outward direction,
wherein the relief pocket is provided in the housing pocket.

17. The medicament delivery device according to claim 16, wherein the medicament delivery member cover lock comprises a locking extension protruding from the distal end of the medicament delivery member cover lock in a perpendicular direction,
wherein the locking extension is configured to be received by the relief pocket.

18. The medicament delivery device according to claim 16, wherein the relief pocket is a slot.
